(19) United States
(12) Patent Application Publication
(10) Pub. No.: US 2017/0049378 A1
(43) Pub. Date: Feb. 23, 2017

(54) Self-Condensing pH Sensor and/or Pepsin Sensor and Catheter Apparatus

(71) Applicants: Jeffery Schipper, San Diego, CA (US); Debra Krahel, San Diego, CA (US); Leo Roucher, San Diego, CA (US)

(72) Inventors: Jeffery Schipper, San Diego, CA (US); Debra Krahel, San Diego, CA (US); Leo Roucher, San Diego, CA (US)

(21) Appl. No.: 14/522,579

(22) Filed: Oct. 23, 2014

Related U.S. Application Data
(63) Continuation-in-part of application No. 11/545,182, filed on Oct. 10, 2006, now Pat. No. 9,005,131.

(51) Int. Cl. A61B 5/00; A61B 5/097; A61B 5/08; A61B 5/145; A61B 1/06

(52) U.S. Cl. CPC A61B 5/4211; A61B 5/0004; A61B 5/14539; A61B 1/0684; A61B 5/6852; A61B 5/7405; A61B 5/742; A61B 5/746; A61B 5/14546; A61B 5/7282; A61B 5/082; A61B 5/097; A61B 5/4233; A61B 2560/0475; A61B 2010/0087

(57) ABSTRACT

The present invention is a system for continuous real-time monitoring of a patient's breath chemistry comprising a plurality of components, including a self-condensing pH sensor distally mounted on a catheter, a pepsin sensor distally mounted on a catheter either separately or in combination with a self-condensing pH sensor, a transmitter with hydration sensing circuitry, and processing receiver/data recorder that can be a smart phone, tablet, TV, watch, wearable device, or custom designed device with wireless capability and utilizing an APP (application) that displays and records the pH and/or pepsin collected data.

### RELATED APPLICATIONS

This application is a continuation-in-part of U.S. patent application Ser. No. 11/545,182 filed on Oct. 10, 2006. All of these applications are incorporated herein by this reference.

### FIELD OF THE INVENTION

The field of art to which this invention relates is in the monitoring of breath chemistry in a patient's airway to provide information that enables physicians to diagnose certain respiratory diseases associated with gastroesophageal reflux (GER). More specifically, the present invention provides for continuous real time monitoring of the pH level of a patient's breath and the pepsin enzyme level of a patient's breath, either simultaneously or separately

### BACKGROUND OF THE INVENTION

Recently, it has been reported that the monitoring of acidity or pH of a patient's breath could help physicians in estimating the potential for and occurrence of asthma, laryngopharyngeal reflux disease (LPRD), aspiration-related lung diseases, chronic obstructive pulmonary disease (COPD), and sleep related breathing disorders such as obstructive sleep apnea (OSA).

Gastroesophageal reflux in the airway is associated with, and known to exacerbate, widespread respiratory diseases such as asthma, laryngopharyngeal reflux disease (LPRD), aspiration-related lung diseases, chronic obstructive pulmonary disease (COPD), and sleep related breathing disorders such as obstructive sleep apnea (OSA). Reflux in the airway is also prevalent in infants and children as well as intubated or sedated patients in whom current pH diagnostic procedures are contraindicated. In extreme cases, the exposure of acid reflux into the respiratory system can lead to aspiration pneumonia or acute respiratory distress.

Given the current state of commercialized products, clinicians are limited in their ability to test pH or pepsin in the respiratory tract. Evaluation of patient pH can be conducted by a qualified physician in a typical 24 hour pH study, using a pH measurement catheter. The presently available pH monitoring and diagnostic devices require insertion of a pH measurement catheter through a patient's nose, past the epiglottis, through the upper esophageal sphincter (UES), and into the esophagus. These catheters are comprised of a pH sensor and reference sensor at the catheter distal end, and require immersion in liquid to function effectively. The devices have invasive or uncomfortable consequences for the patient. Because they pass through the larynx and into the esophagus, discomfort during swallowing, talking, and movement can occur. A recently introduced product, the Medtronic Bravo™, is a catheter based device that requires attachment of a pH measurement capsule to the esophagus wall. The product requires a larger diameter trans-nasal catheter to place the capsule. Because these methods are invasive and uncomfortable, only a small percentage of prospective patients are able to undergo pH monitoring.

Pepsin is an enzyme derived from pepsinogen which is released by the chief cells in the stomach. This enzyme functions to degrade food proteins into peptides. In the stomach lining, parietal cells release pepsinogen which is activated by hydrochloric acid (HCl). A gastric hormone and the vagus nerve trigger the release of both pepsinogen and HCl from the stomach lining when food is ingested. Hydrochloric acid creates an acidic environment, which allows pepsinogen to unfold and cleave itself in an autocatalytic fashion, thereby generating the enzyme pepsin in an active form. Pepsin will digest approximately twenty percent of exposed amide bonds by cleaving after the N-terminal of aromatic amino acids such as phenylalanine, tryptophan, and tyrosine. Pepsin is primarily active in acidic environments between the temperature range of 37° C. and 42° C. Its primary site of synthesis and activity is in the stomach where the pH is 1.5 to 2. Pepsin exhibits maximal activity at pH 2.0 and is inactive at pH 6.5 and above. Pepsin becomes fully denatured or irreversibly inactivated when exposed to a pH 8.0 or above. But below pH 8.0, pepsin can be reactivated upon re-acidification.

The stability of pepsin at high pH has significant implications on disease attributed to laryngopharyngeal reflux. Pepsin travels up to the larynx following a gastric reflux event. At the mean pH of the laryngopharynx (pH=6.8) pepsin would be inactive but could be reactivated upon subsequent acid reflux events resulting in damage to local tissues.

Pepsin is one of the primary causes of mucosal damage during laryngopharyngeal reflux. When a gastric reflux event occurs, pepsin travels up to the larynx where the environment is approximately pH 6.8. While pepsin is enzymatically inactive in this non-acidic environment, pepsin will remain stable and could be reactivated upon subsequent acid reflux events. Exposure of laryngeal mucosa to enzymatically active pepsin, but not irreversibly inactivated pepsin or acid, results in reduced expression of protective proteins and thereby increases laryngeal susceptibility to damage.

It is known that pepsin may also cause mucosal damage during weakly acidic or non-acid gastric reflux. Such exposure to pepsin at neutral pH and endoyctosis of pepsin causes changes in gene expression associated with inflammation, which conceals signs and symptoms of active reflux. Pepsin in esophageal airway is considered to be a sensitive and specific marker for laryngopharyngeal reflux. Therefore, there is a need to develop new pepsin-targeted diagnostic tool(s for gastric reflux. A rapid but non-continuous pepsin diagnostic test called Peptest is available which determines the presence of pepsin in saliva samples. The disadvantage of the Peptest is that the single saliva test can miss reflux events. Continuous real-time monitoring capability would be a useful tool for the physician to effectively diagnose reflux events in patients.

Placement of esophageal catheters requires special expertise to identify the physical landmarks required for proper catheter placement. Typically, pressure measurements are conducted to find the lower esophageal sphincter and upper esophageal sphincter, with endoscopic confirmation of placement required in some cases.

Traditional pH catheters used to conduct measurements of pH in the patient's laryngeal region and have several limitations when placed in the upper airway. They are capable of only measuring liquid reflux events which extend past the UES. They are subject to becoming fouled, contaminated or embedded in the mucosal wall. If placed higher in the airway, the sensor can become dehydrated, losing electrical continuity with the reference electrode. In these cases, the accuracy and reliability of the pH measurements are compromised.

Accordingly, there is a need for a novel monitoring system with electronic or wireless communication linked to a processing receiver with data recording capability that can continuously measure and record pH or pepsin or both simultaneously in the patient's airway in real time.

### SUMMARY OF THE INVENTION

The present invention pertains to a device for monitoring the breath chemistry of a patient's exhaled breath in real-time. The present invention is a system comprising a self-condensing pH and/or a pepsin sensor distally mounted on a catheter, a transmitter with hydration sensing circuitry for the pH sensor or pepsin sensor, and processing receiver/data recorder. The processing receiver/data recorder compromises typical cell phones, smart phones, or custom designed apparatus which includes touchscreen mobile wireless devices such as PDAs, tablets (e.g. refers to all current and future variants, revisions and generations of the Apple IPAD, Samsung Galaxy, HP, Acer, Surface, Nook, Google Nexus, Sony, Kindle and all future tablets manufactured by these and other manufactures), related handheld devices including Apple IPOD Touch, or wearable timepieces or fob watches and other similar apparatus with WIFI and wireless capability, and remote computers and controllers with wireless connectivity that can utilize an APP (application) to display information during an ambulatory pH or pepsin study.

The self-condensing pH sensor and/or pepsin sensor is located on the distal end of a tubular catheter designed to be inserted trans-nasally into the patient's upper airway, and more specifically, into the oropharynx region of a patient's upper airway. The catheter has at least one lumen that extends along the longitudinal length of the catheter. The self-condensing pH sensor uses the catheter shaft as its outer tubular member to house a silver chloride reference element, an ion conducting path, and an antimony sensor element isolated in an inner tubular member that is co-linearly or coaxially configured within the catheter tubular member. The performance of the self-condensing sensor may be enhanced by including a hygroscopic coating. The pepsin sensor uses the catheter shaft as its outer tubular member to house a sensor containing a substrate and an active component for detecting the presence of pepsin enzyme. A separation means may be employed in close proximity to the pH and/or pepsin sensor to keep the pH and/or pepsin sensor from directly contacting the mucosal tissue of the patient's oropharynx region, to prevent the pH and pepsin sensor from becoming entrapped in the airway mucosal wall which could impair the sensor's ability to measure the airway pH and pepsin. An optional lighting source is also located in the distal end of the catheter to simplify placement of the self-condensing sensor in the oropharynx region. The optional lighting of the present invention addresses catheter insertion and location with a innovative method of placement, using a continuous or flashing light emitting diode (LED) embedded in the distal end of the catheter to provide a visual sighting means for the physician.

A transmitter with an antenna is located at the proximal end of the catheter and transfers the observed pH and/or pepsin data by employing one of many wireless methods, such as radio-frequency (RF) energy. Alternately, the transfer of observed pH and pepsin data is accomplished by direct wire methods. The transmitter also includes a means to evaluate the signal strength from the pH and/or pepsin sensor to determine whether the sensor is hydrated sufficiently to accurately measure pH and/or pepsin. This is accomplished by periodically analyzing the voltage signal from the pH and/or pepsin sensor to a predetermined set of wave forms.

The pH and/or pepsin data is transferred or updated at specific intervals, which can be varied according to the patient's needs, to a processing receiver that has the capability to record the pH and/or pepsin data on a continuous real-time basis over a specified period of time.

The processing receiver/data recorder can include a removable data card that stores the recorded pH and/or pepsin data for subsequent analysis. The processing receiver/data recorder can also include a visual or auditory alarming means that is generated upon when predetermined pH and/or pepsin values are detected.

In operation, the distally mounted pH and/or pepsin sensor/catheter assembly is inserted through one of the patient's nostrils until the self-condensing pH and/or pepsin sensor is positioned in the oropharynx region, typically above the epiglottis. When the pH and pepsin sensor is located in the proper position, a securing means is used to fasten the pH and/or pepsin sensor/catheter assembly in place.

These and other features, aspects and advantages of the present invention will become better understood with reference to the following descriptions and claims.

### BRIEF DESCRIPTION OF THE DRAWINGS

FIG. 1 is a perspective representation of the present invention system, showing the proximal end of the catheter exiting one nostril and draped over the ear of a typical patient, and a wireless transmitting device which communicates with a processing receiver/data recorder.

FIG. 2 is cross-sectional representation of the present invention system, showing the location of the distal end of the catheter comprising a self condensing pH and pepsin sensor positioned adjacent to the patient's uvula.

FIG. 3 is a representation of the entire catheter length with a self condensing pH or pepsin sensor and separation means located on the distal end of the catheter and a transmitting device located on the proximal end of the catheter.

FIG. 4 is a partially sectional side view of the self condensing pH or pepsin sensor demonstrating, in detail, the orientation and components of the pH sensing means, including the position of the reference wick surrounding an inner collinearly positioned tubular member containing the antimony sensor.

FIG. 5 is one embodiment of the separation means comprising a sectional view of a tear-drop shaped structure mounted on the exterior surface of the distal end of the catheter, wherein the self-condensing pH sensor is mounted flush with said distal end of said catheter and a light emitting diode (LED) is mounted in the tip for illumination.

FIG. 6 is one embodiment of the separation means comprising a tear-drop shaped structure mounted on the exterior surface of the distal end of the catheter, wherein the self condensing pH and pepsin sensor are recessed within said distal end of said catheter and a light emitting diode (LED) is mounted in the tip for illumination.

FIG. 7 is one embodiment of the pepsin sensor location within the tear-drop shaped tip structure mounted on the exterior surface of the distal end of the catheter, wherein the pepsin sensor is comprised of a substrate and an active component for detecting the presence of pepsin enzyme.

FIG. 8 is one embodiment of the pepsin sensor located within the distal end of the catheter utilizing an access port to the oropharyngeal environment in order to sample for the presence of pepsin.

### DESCRIPTION OF THE PREFERRED EMBODIMENTS

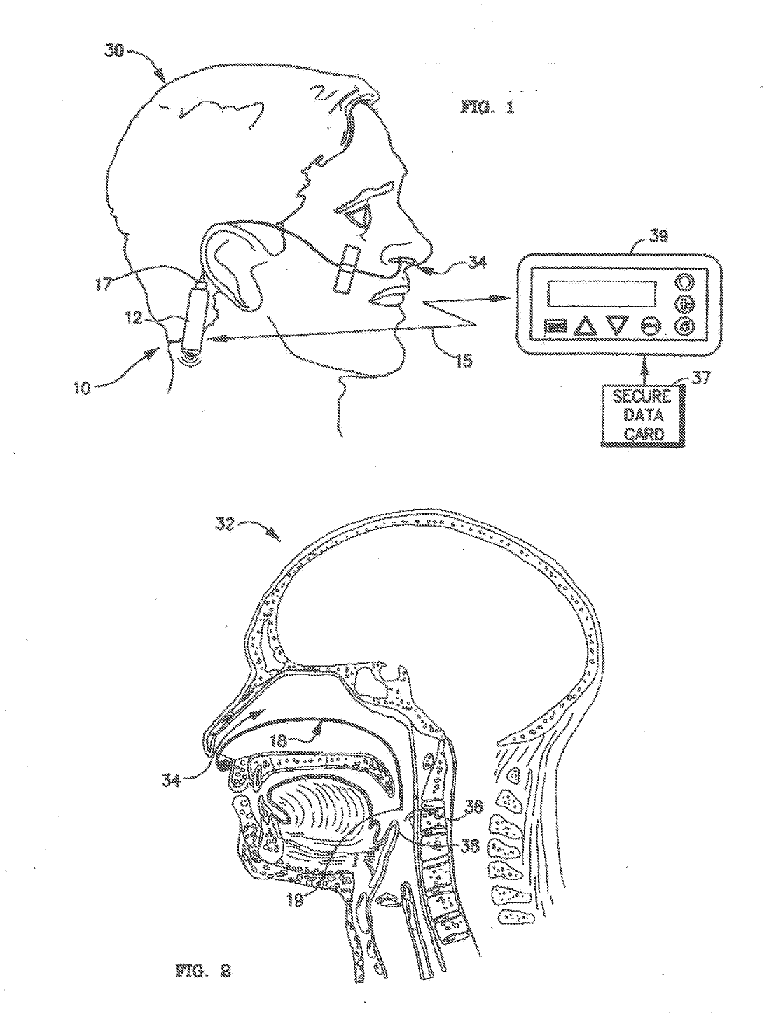

The present invention is a system comprising a plurality of components, including a self-condensing pH and/or pepsin sensor distally mounted on a catheter, a transmitter with hydration sensing circuitry for the pH and/or pepsin sensor, and processing receiver/data recorder. Referring to FIG. 1, a perspective representation of the present invention system 10 is shown where the catheter 18 is exiting one nostril 34 and draped over the ear of a typical patient 30. The proximal end 17 of the catheter 18 is connected to a transmitting device 12, which communicates with a processing receiver with data recording capability 39 (herein referred to the “processing receiver/data recorder” 39). The preferred method of communication is by wireless means 15, however, it is anticipated by the Applicants that the transmitting device can communicate with the processing receiver/data recorder 39 by a direct wired means. The wireless transmitter device 12 incorporates an antenna that transfers the measured pH and pepsin data by employing one of many wireless methods, such as radio-frequency (RF) energy. Other methods of wireless transmitting include optical and infrared means and Bluetooth™ technology.

The processing receiver/data recorder 39 is designed as the operator interface between both the clinician and patient, and a means for recording pH and pepsin data and user events during an ambulatory study. The processing receiver/data recorder 39 is typically battery powered, include a clock to keep and display time, memory to store patient data, buttons for recording patient events, and a connection to a pH and pepsin sensor/amplifier front end. This connection can be wired or wireless. Additionally, the recorder typically provides a way to upload the data to a PC for storage and analysis. The processing receiver/data recorder 39 includes low power microprocessors such as Microchip model 16F and 18F series controllers, and the ATMEL 8051 family of devices. Timekeeping operations are accomplished by the microprocessor, or alternately accomplished by a dedicated time chip such as the Dallas DS1338 real time clock. To keep power consumption to a minimum, LCD displays such as the Optrex DMC-16204 is utilized. Wireless communication can be accomplished in a variety of means, from very simple frequency shift keying techniques to technically advanced spread spectrum designs.

It is anticipated by the Applicant that the processing receiver/data recorder can compromise a typical cell phone, smart phones, or similar apparatus includes all remote cellular phones using channel access methods (with cellular equipment, public switched telephone network lines, satellite, tower and mesh technology), mobile phones, PDAs, tablets (e.g. refers to all current and future variants, revisions and generations of the Apple IPAD, Samsung Galaxy, HP, Acer, Microsoft, Nook, Google Nexus, Sony, Kindle and all future tablets manufactured by these and other manufactures), Apple IPOD Touch, or a television, timepiece or fob watch and other similar apparatus with WIFI and wireless capability, and remote computers and controllers with wireless connectivity that utilizes an APP (application) that can display or provides a means for recording pH and pepsin data and user events during an ambulatory study.

The processing receiver/data recorder can include a removable data card that stores the recorded pH and pepsin data for subsequent analysis. The processing receiver/data recorder can also include a visual or auditory alarming means that is generated upon when predetermined pH and pepsin values are detected.

Before an ambulatory pH and pepsin study is started, the clinician typically calibrates the pH sensor with calibration buffer solutions of know values. The processing receiver/data recorder 39 prompts the user on what to do, interprets the digital signal output from the sensor/transmitter while in the various buffer solutions, calculates calibration factors and stores them in non-volatile memory. Other parameters such as time/date adjustment, study duration, and display options can also be adjusted at this time.

After successful calibration is completed and the clinician has setup the unit to their satisfaction, the processing receiver/data recorder 39 is given to the patient 30 and instructed on its use. The front of the processing receiver/data recorder 39 has a multitude of buttons that are pressed by the patient 30 to record symptoms and activities. Symptoms such as heartburn, and coughing are recorded at the time the patient feels the onset of these events and will be compared to the pH and/or pepsin values recorded during these time periods. Activities such as meals, supine (laying down) are also logged and used by the physician to assist in making a proper diagnosis.

The processing receiver/data recorder 39 includes a removable data card 37 that stores the measured pH and pepsin data for subsequent analysis. Once a removable data card 37 with stored data is removed, a new removable data card can be inserted for recording the next patient study of pH and pepsin data. The processing receiver/data recorder 39 can also include outputs to other data recording devices, such as equipment used in hospitals and sleep clinics.

The processing receiver/data recorder 39 also includes software that is specifically designed to analyze waveforms generated by the waveform input hydration sensing circuitry located in the transmitter 12. The software or APP is programmed to initiate a visual or auditory alarm and/or stop recording pH and pepsin data upon the occurrence of unreliable waveforms. The processing receiver/data recorder 39 can also incorporate an alerting means that is initiated when certain pH and pepsin parameters are measured. For example, if the pH and pepsin enters a range known to be associated with a particular respiratory disease, a visual or auditory alarm can be generated.

FIG. 2 is a cross-sectional representation of a typical patient 32 with the present invention system 10, properly inserted through the nasal passages 34. The distal end of the catheter 19 containing a self condensing pH and/or pepsin sensor 20 is positioned above the epiglottis 38. The positioning of the pH and/or pepsin sensor 20 is important for monitoring real-time pH and pepsin while providing comfort for the patient and not interfering with other patient functions such as swallowing and talking. Methods for verifying the position of the distal end of the catheter in the patient's oropharynx region 36 include calibrated markings on the catheter shaft. The sensor position can also be visually established. To simplify placement, another embodiment includes in the distal end 19 of the catheter 18 an embedded light source, such as a light emitting diode (LED), which can be illuminated continuously or in a flashing mode to aid in positioning the distal pH and/or pepsin sensor 20 in the oropharynx region 36 of a cross-sectional 32 patient 30.

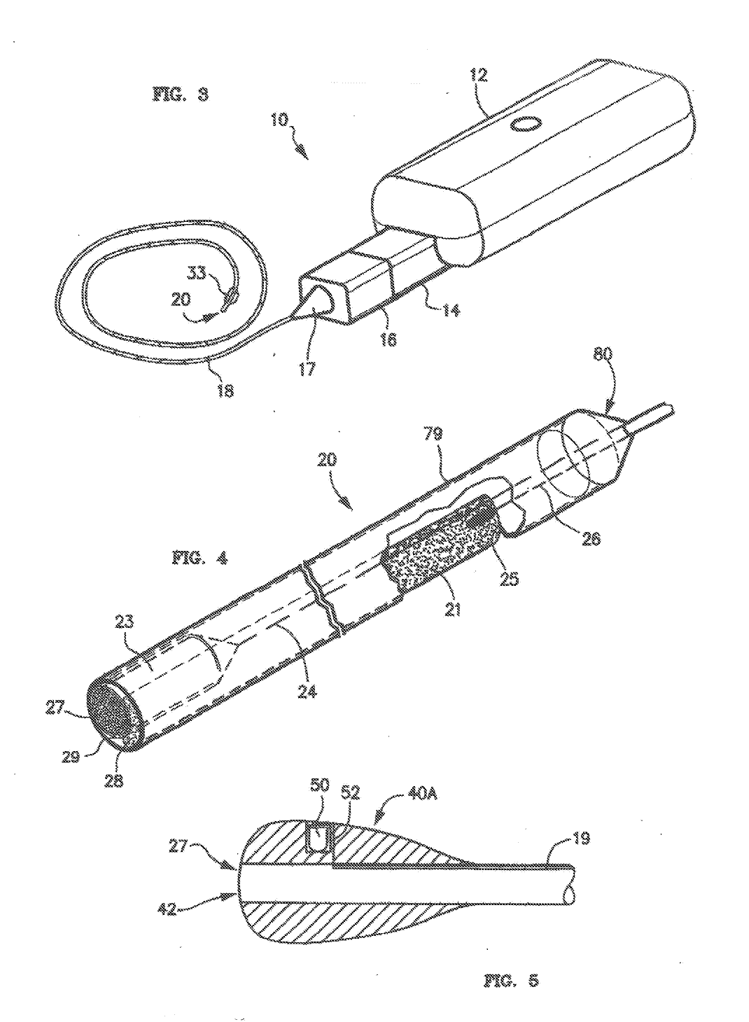

FIG. 3 is a representation of the entire catheter length with a self condensing pH and pepsin sensor 20 and separation means 33 located on the distal end of the catheter 19 and a transmitting device 12 located on the proximal end 17 of the catheter 18. A connector 16 on the catheter is shown engaged to a receiving connector 14 on the transmitting device 12. The catheter 18 can be a single or a multi-luminal design for allowing electrical connection from the distal pH and pepsin sensor to extend throughout the longitudinal length of the catheter and terminate in the transmitter 12, located on the proximal end 17 of catheter 18.

The transmitter 12 located on the proximal end 17 of the catheter 18 contains an electrical circuit to communicate, preferably wirelessly 15 (as shown in FIG. 1), pH and pepsin information to a processing receiver/data recorder 39. The transmitter 12 comprises a circuit board populated with various discrete and semiconductor components and mounted in a housing. The housing is generally fabricated from a polymeric material such as, polycarbonate, acrylic, polysulfone, polyethylene, polypropylene, polystyrene, ABS, nylon, delrin, or polyurethane composites. A connector 14 is designed to engage with a connector 16 located on the proximal end 17 of the catheter 18. A means can be incorporated into the housing which allows the housing to be recoverably secured to the patient or simply draped over a patient's ear as shown in FIG. 1. It is also anticipated by the Applicants that the transmitter 12 can be connected to the processing receiver/data recorder using typical hard wiring techniques.

In the wireless design, the transmitter 12 receives input from the pH and/or pepsin sensor on a continuous real time basis and sends this analog data, at a specified frequency, to a remotely located processing receiver/data recorder 39. The wireless transmitter device 12 incorporates an antenna that transfers the pH and/or pepsin data using wireless methods, such as radio-frequency (RF), optical and infrared means. The antenna can extend externally from the transmitter housing or can be concealed inside the housing.

The transmitter 12 also contains circuitry 70 to interrogate the hydration level of the pH. The hydration monitoring circuitry 70 is shown in more detail in FIG. 12.

FIG. 4 is a partially sectional side view of the self-condensing pH 20 demonstrating in detail the orientation and components of the pH and pepsin sensing means 20, including the position of the reference wick 28 surrounding an inner co-linearly positioned tubular member containing the antimony element 23.

The self-condensing pH and/or pepsin sensor 20 is located within the tubular member of catheter 18 and is either co-linearly or coaxially aligned within the outer tubular member. The pH and pepsin sensor also includes an inner tubular member 29 that is usually fabricated by an extrusion or dip coating process using a variety of polymeric materials including polyimide, polyethylene, polypropylene, polyvinyl chloride, polyether block amide, polystyrene, ABS, nylon, delrin, polyethylene terephthalate (PET), fluorinated ethylene-propylene (FEP) or polytetrafluoro-ethylene (PTFE). The inner tubular member 29 has an outside diameter smaller than the inside diameter of the outer catheter and generally is in the range of 0.015″ to 0.030″, and preferably between 0.020″ and 0.028″. Its wall thickness is typical for its diameter and generally is in the range of 0.00025″ to 0.002″ and preferably between 0.0005″ and 0.001″.

Located within the inner tubular member 29 is an antimony element 23 having a surface area at the terminal end 27. The antimony element 23 is generally 99% pure and free from significant contaminants. The Applicant contends that the antimony element 23 could be replaced with other metallic substances similar to antimony which exhibit a change in electrical potential when immersed in different pH and pepsin fluids. Furthermore, other potential sensor elements such as specially formulated polymers, semiconductor technology, Ion Sensitive Field Effect Transistors (“ISFET's), optical sensing, capacitive sensing, and nanotechnology could be employed.

The antimony element 23 is engaged at its proximal end to an electronic communication means 24. Typically the electronic communication means comprises electrical wire that has an internal core comprising an electrically conductive metallic material, which is encased by a nonconductive jacket. The means of engagement typically employs standard soldering technology and can be supported by a variety of means to provide strain relief. The terminal surface 27 of the antimony element 23 defines the distal terminal boundary of the sensor 20 and is the surface that is exposed to liquid or humid gaseous environments. As shown in FIG. 4, the antimony element 23 and the reference wick 28 are substantially in the same plane. However, it is anticipated by the Applicants that several designs or embodiments in which the antimony element and reference electrode are not substantially in the same plane. For example, a coaxial design in which the antimony element, protruding beyond the center of the sensor terminal end, has the advantage of providing a greater surface area of antimony element to react with the condensing sample. In addition, a co-linear sensor in which the antimony sensor protrudes past the plane of the wick and the extension that is angled towards the wick provides the advantage of providing a greater surface area but additionally diminishes or reduces the contact angle between the antimony element and reference wick. Reducing the angle between the wick and the antimony element may provide a more reliable measurement in low humidity conditions. Still another design or embodiment entails either the coaxial or co-linear design, where the antimony element is recessed from the plane of the wick. This design has the potential for greater stability due to the larger film thickness that condenses and resides on the antimony face. In addition, a further benefit of this design is the potential for greater sensitivity in sleep apnea clinical conditions due to the increased angle between the electrodes at the top of the recess. This increased angle may cause the surface tension to break contact between the electrodes more rapidly than on a planar design in the event of a decrease in fluid deposition.

The performance of the sensor 20 may be enhanced in some environments by the inclusion of a coating (not shown) on this distal surface. One example would be a hygroscopic coating to enhance the absorption and retention of moisture on the sensor in humidified gases and aerosols. Materials such as hydrophilic polyurethanes, polyacrylamides, poly(2-hydrox-ethyl-methacrylate), other methacrylate copolymers, perfluorinated polymers, polysaccharides, polyvinylchloride polyvinyl alcohol and silicones could all be utilized as surface enhancements either alone, in combination, or with modifications.

The use of a hygroscopic coating may also enable the use of other sensor configurations by enhancing the ability of the reference and pH and pepsin elements to remain in contact through periods of little or no fluid contact. One example is a side mounted reference sensor in close proximity to a pH sensing element located on the sensor end. A second design of the pH sensor which would benefit from a coating in this application includes reference and sensing elements placed on the sides of the catheter tube either opposed or linearly. The coating may also provide benefits by maintaining continuity between multiple sensors and a single reference.

Located proximally, the pH sensor ranges from 1-10 millimeters from the proximal end of the antimony element 23 and preferably 4-5 millimeters is a reference element 25. Said reference element 25 is primarily composed of a silver core surrounded with a coating of silver chloride. A technology of dipping a silver core in a high temperature bath of silver chloride to produce the silver chloride coating is employed in the present invention. Other means of producing the silver chloride coating exist, including electro-deposition and vapor deposition. The resulting coating generally is 0.0001″ to 0.010″ in thickness, and preferably 0.001″ to 0.005″. The reference element 25 is engaged to an electrical communication means 26, e.g. typical wire that extends to the proximal end 17 of the outer tubular member or catheter 18 and can terminate in a typical electrical connector 16. An adhesive or polymer plug can be placed in a proximal position to the reference element 25 that is engaged to the outer tubular member or catheter 18 which provides support for electrical communication means 24,26 and provides proximal sealing of the outer tubular member or catheter 18.

A reference wick 28 is located between the inside surface of the outer tubular member of the self-condensing pH and pepsin sensor 20 and the outer surface of the inner tubular member 29. In one embodiment the inner tubular member 29 is coaxially offset with the outer tubular member. The reference wick 28 partially surrounds the inner tubular member 29 where the area of the offset coaxial design is large enough to contain the fabric or mesh configuration of the reference wick 28. As discussed in more detail below, the reference wick 28 has a mesh or fibrous configuration which functions to entrain or retain an ion conducting fluid 21.

Reference wick 28 is physically separated from the antimony element 23 by the wall of the inner tubular member 29. It is important to the present invention that the reference wick 28 does not engage or contact the antimony element 23 at any point. The reference wick 28 can be fabricated from a variety of polymeric based materials. Examples of such materials are polysaccharides, polyester, polyethylene, polypropylene, polyvinyl chloride (PVC), polystyrene, ABS, nylon, delrin, polyethylene terephthalate (PET), polytetrafluoroethylene (PTFE), collagen, Hytrel (thermoplastic polyester elastomer), or any material or combination of materials which exhibit a weave, felt or mesh design that facilitates wicking or ion conduction. One example of a preferable material for the reference wick 28 is a polyester fabric mesh. The reference wick 28 functions similarly to a plurality of capillary tubes which facilitate the transport of ions between the antimony element 27 and reference element 25.

The reference wick 28 is impregnated with an ion conduction fluid 21. Typical conduction fluids include those that contain sodium chloride or potassium chloride and water. One example that can be used with the sensor is a saturated aqueous solution of sodium chloride containing from 1-10 percent polysaccharide, with a preferred range of 1-3 percent. Other materials that can function as the reference wick 28 with an ion conduction fluid 21 include ion carrying gels, hydrogels, open cell foams and porous frits of various materials. These gels, hydrogels, and other materials aid in reducing the diffusion of contaminants into the ion conduction fluid.

FIG. 5 is one embodiment of the present invention with separation means 40a comprising a cross section of the tear-drop shaped structure 43 mounted on the exterior surface of the distal end 19 of the catheter 18. The tear-drop shaped structure 43 is designed to provide separation means when the catheter 18 and distally mounted pH and/or pepsin sensor 20 are positioned above the epiglottis 38. In this embodiment, the self condensing pH and/or pepsin sensor 20 is mounted flush 42 with said distal end 27 of the catheter 18. The tear-drop shaped structure 43 is adhered to the outside surface of the catheter 18 using general adhesive technology. The distal end of the tear-drop shaped structure 43 generally has an outside diameter in the range of 0.040″ to 0.250″, and preferably between 0.100″ and 0.150″. The outside diameter then slopes towards the proximal end of the tear-drop shaped structure 43 where it approximates the outside diameter of the catheter 18. The tear-drop shaped structure 43 is usually fabricated by machining or molding means using a variety of polymeric materials including polyimide, polyethylene, polypropylene, polyvinyl chloride, epoxy, polyurethane, polycarbonate, acrylic, polystyrene, ABS, nylon, delrin, polyethylene terephthalate (PET), polyether block amide, fluorinated ethylene-propylene (FEP) or polytetrafluoro-ethylene (PTFE). As shown in this FIG. 5, to simplify placement within the oropharynx region of a patient, an embedded light source 50 is included in close proximity to the distal end 19 of the catheter 18. The light source 50 is connected to an electrical wiring means 52 that extends the length of the catheter, incorporated as an element of the connectors 14, 16 and is connected to a power source in the transmitter 12. The embedded light source 50 preferably is comprised of a light emitting diode (LED), which can be illuminated continuously or in a flashing mode to aid in determining the location of the distal pH and pepsin sensor 20 in the oropharynx region 36.

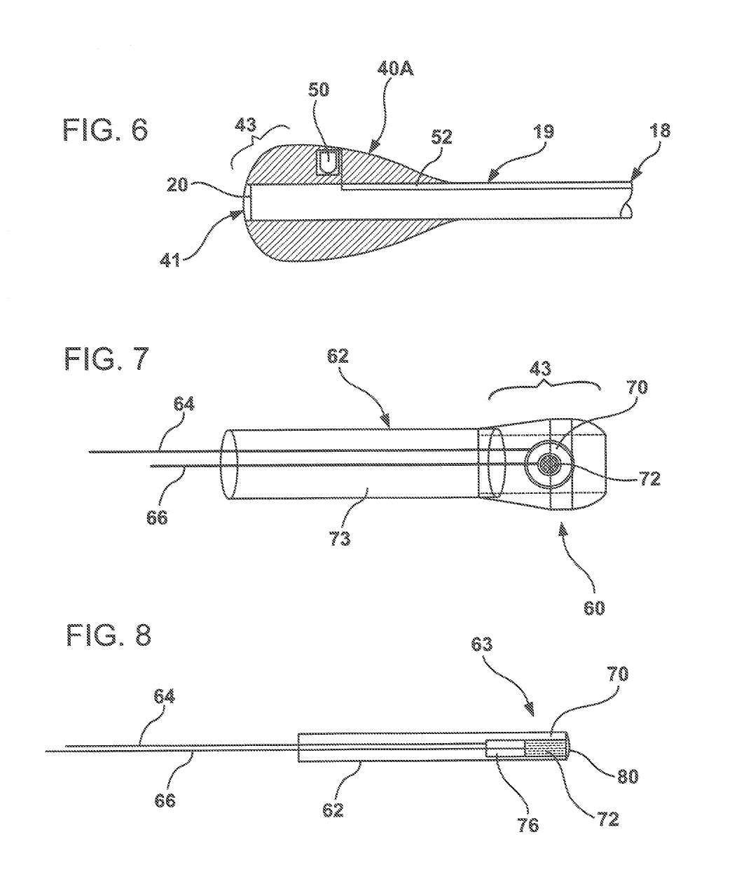

FIG. 6 is another embodiment of the present invention with separation means 40a comprising a cross section of the tear-drop shaped structure 43 mounted on the exterior surface of the distal end 19 of the catheter 18. The tear-drop shaped structure 43 is designed to provide separation means when the catheter 18 and distally mounted pH and/or pepsin sensor 20 are positioned above the epiglottis 38. In this embodiment, the self condensing pH and/or pepsin sensor 20 is recessed 41 in the range of 0.005″ to 0.020″, and preferably 0.010″ to 0.015″ within said distal end of said catheter. The tear-drop shaped structure 43 is adhered to the outside surface of the catheter 18 using general adhesive technology. The distal end of the tear-drop shaped structure 43 generally has an outside diameter in the range of 0.040″ to 0.0250″, and preferably between 0.100″ and 0.150″. The outside diameter then slopes towards the proximal end of the tear-drop shaped structure 43 where it approximates the outside diameter of the catheter 18. A tear-drop shaped structure 43 is usually fabricated by a machining or molding process using a variety of polymeric materials including polyimide, polyethylene, polypropylene, polyvinyl chloride, epoxy, polyurethane, polycarbonate, acrylic, polystyrene, ABS, nylon, delrin, polyethylene terephthalate (PET), polyether block amide, fluorinated ethylene-propylene (FEP) or polytetrafluoro-ethylene (PTFE). As shown in this FIG. 6, to simplify placement within the oropharynx region of a patient, an embedded light source 50 is included in close proximity to the distal end 19 of the catheter 18. The light source 50 is connected to an electrical wiring means 52 that extends the length of the catheter, incorporated as an element of the connectors 14, 16 and is connected to a power source in the transmitter 12. The embedded light source 50 preferably is comprised of a light emitting diode (LED), which can be illuminated continuously or in a flashing mode to aid in determining the location of the distal pH and pepsin sensor 20 in the oropharynx region 36.

FIG. 7 is one embodiment of the pepsin sensor location within the tear-drop shaped tip structure mounted on the exterior surface of the distal end of the catheter, wherein the pepsin sensor is comprised of a substrate and an active component for detecting the presence of pepsin enzyme.

FIG. 8 is one embodiment of the pepsin sensor located within the distal end of the catheter utilizing an access port to the oropharyngeal environment in order to sample for the presence of pepsin.

By way of example, in clinical operation of the present invention, the physician generally applies a medicament to anesthetize a patient's nasal passages before inserting the self-condensing pH and/or pepsin sensor and catheter apparatus 10. The clinician will then position the sensor 20 located at the distal end 19 of the catheter 18 so that is positioned in the oropharynx region adjacent to the uvula. In this position, the patient does not feel discomfort during normal activities such as talking, eating, or drinking. The proximal end 17 of the catheter 18 is then secured to the patient's face with tape or other means to ensure that it stays in the appropriate position.

During the pH and/or pepsin measurement study, the patient can wear the processor receiver/data recorder 39 with a provided carrying case or alternately it can be place in a convenient location within the room where the patient resides. The transmitter 12 can be releasably attached to the patient with tape or other appropriate means. The patient continues to wear the inserted catheter 18 with self-condensing sensor 20 for the duration of the study.

The patient is instructed to press the corresponding button on the processing receiver/data recorder when any of the following events occur during the study period:
a. Cough (press at the onset of the symptom; if it lasts for a long time, they press the button again after the symptom has stopped)
b. Eat a meal or snack; drink a beverage (press button when they begin and when they finish eating or drinking).
c. Lie down in a supine position (when they lie down and again when they get up).
d. Experience chest pain or heartburn (press at the onset of the symptom; if it lasts for a long time, they press the button again after the symptom has stopped).
e. Other events such as throat clearing, globus (difficulty swallowing), post nasal drip, and various predefined symptoms which can be programmed into the processing receiver/data recorder during initial setup.

We claim:

1. An apparatus for monitoring of pH, said apparatus comprising:
a catheter apparatus having a distal end, a proximal end, and at least one lumen that extends along the longitudinal length of said catheter apparatus and communicating with said distal end and said proximal end;
a self-condensing pH sensor located in close proximity to said distal end, said self-condensing pH sensor designed to be positioned in the upper airway of a patient;
a data transmitter attached to said catheter proximal end;
said self-condensing sensor in electrical communication with said data transmitter; and
a processing receiver/data recorder in communication with said data transmitter, said processing receiver/data recorder compromises typical cell phones, smart phones, or custom designed apparatus which includes touchscreen mobile wireless devices such as PDAs, tablets (e.g. refers to all current and future variants, revisions and generations of the Apple IPAD, Samsung Galaxy, HP, Acer, Surface, Nook, Google Nexus, Sony, Kindle and all future tablets manufactured by these and other manufactures), related handheld devices including Apple IPOD Touch, or wearable timepieces or fob watches and other similar apparatus with WIFI and wireless capability, and remote computers and controllers with wireless connectivity that can utilize an APP (application) that can display or provides a means for recording pH data and user events during an ambulatory study.

2. The apparatus for monitoring of pH as recited in claim 1, wherein said catheter has separation means to restrain said self-condensing sensor element from directly contacting the airway mucosal membranes of a patient, said separation means located near said distal end of said catheter.

3. The apparatus for monitoring of pH as recited in claim 2, wherein said separation means comprises a tear-drop shaped structure.

4. The apparatus for monitoring of pH as recited in claim 1, wherein said processing receiver/data recorder is in wireless communication with said data transmitter.

5. The apparatus for monitoring of pH as recited in claim 1, wherein said wireless communication is conducted continuously in real-time.

6. The apparatus for monitoring of pH as recited in claim 1, wherein said processing receiver/data recorder is in continuous real-time wired communication with said data transmitter.

7. The apparatus for monitoring of pH as recited in claim 1, wherein said processing receiver/data recorder has the capability to analyze the hydration level of said self-condensing pH sensor.

8. The apparatus for monitoring of pH as recited in claim 1, further comprising a light source mounted on said catheter in close proximity to said distal end.

9. The apparatus for monitoring of pH as recited in claim 8, wherein said light source is a light emitting diode.

10. The apparatus for monitoring of pH as recited in claim 9, wherein said light source functions to facilitate the catheter insertion, placement and location of said pH sensor within said oropharynx area of a patient, said light source comprising a continuous or flashing light emitting diode (LED) embedded in the distal end of the catheter for providing a visual sighting means for the physician.

11. The apparatus for monitoring of pH as recited in claim 1, further comprising a removable data storage medium, said removable data storage medium designed to communicate with said processing receiver/data recorder, said removable data storage medium further designed to store recorded pH measurements monitored by said self-condensing pH sensor over a period of time.

12. The apparatus for monitoring of pH as recited in claim 1, wherein said processing receiver/data recorder includes a visual or auditory alarming means that is generated upon the occurrence of a certain pH range.

13. An apparatus for monitoring of pepsin, said apparatus comprising:
a catheter apparatus having a distal end, a proximal end, and at least one lumen that extends along the longitudinal length of said catheter apparatus and communicating with said distal end and said proximal end;
a pepsin sensor located in close proximity to said distal end, said pepsin sensor designed to be positioned in the upper airway of a patient either separately or in combination with a self-condensing pH sensor;
a data transmitter connected to said proximal end;
said pepsin sensor in electrical communication with said data transmitter; and
a processing receiver/data recorder in communication with said data transmitter, said processing receiver/data recorder compromises typical cell phones, smart phones, or custom designed apparatus which includes touchscreen mobile wireless devices such as PDAs, tablets (e.g. refers to all current and future variants, revisions and generations of the Apple IPAD, Samsung Galaxy, HP, Acer, Surface, Nook, Google Nexus, Sony, Kindle and all future tablets manufactured by these and other manufactures), related handheld devices including Apple IPOD Touch, or wearable timepieces or fob watches and other similar apparatus with WIFI and wireless capability, and remote computers and controllers with wireless connectivity that can utilize an APP (application) that can display or provides a means for recording pepsin data and user events during an ambulatory study.

14. The apparatus for monitoring of pepsin as recited in claim 13, wherein said catheter has separation means to restrain said pepsin sensor element from directly contacting the airway mucosal membranes of a patient, said separation means located near said distal end of said catheter.

15. The apparatus for monitoring of pepsin as recited in claim 14, wherein said separation means comprises a tear-drop shaped structure.

16. The apparatus for monitoring of pepsin as recited in claim 13, wherein said processing receiver/data recorder is in wireless communication with said data transmitter.

17. The apparatus for monitoring of pepsin as recited in claim 13, wherein said wireless communication is conducted continuously in real-time.

18. The apparatus for monitoring of pepsin as recited in claim 13, wherein said processing receiver/data recorder is in continuous real-time wired communication with said data transmitter.

19. The apparatus for monitoring of pepsin as recited in claim 13, further comprising a light source mounted on said catheter in close proximity to said distal end.

20. The apparatus for monitoring of pepsin as recited in claim 19, wherein said light source is a light emitting diode.

21. The apparatus for monitoring of pepsin as recited in claim 20, wherein said light source functions to facilitate the catheter insertion, placement and location of said pepsin sensor within said oropharynx area of a patient, said light source comprising a continuous or flashing light emitting diode (LED) embedded in the distal end of the catheter for providing a visual sighting means for the physician.

22. The apparatus for monitoring of pepsin as recited in claim 13, further comprising a removable data storage medium, said removable data storage medium designed to communicate with said processing receiver/data recorder, said removable data storage medium further designed to store recorded pepsin measurements monitored by said pepsin sensor over a period of time.

23. The apparatus for monitoring of pepsin as recited in claim 13, further comprising a measurement system that can include a means to activate an auditory and/or visual alarm when predetermined pepsin values are detected.

24. An apparatus for monitoring of pH and pepsin, said apparatus comprising:
a catheter apparatus having a distal end, a proximal end, and at least one lumen that extends along the longitudinal length of said catheter apparatus and communicating with said distal end and said proximal end;
a self-condensing pH sensor and pepsin sensor located in close proximity to said distal end, said self-condensing pH sensor and pepsin sensor designed to be positioned in the oropharynx area of a patient;
a data transmitter connected to said proximal end;
said self-condensing pH sensor and pepsin sensor in electrical communication with said data transmitter; and a processing receiver/data recorder in communication with said data transmitter, said processing receiver/data recorder compromises typical cell phones, smart phones, or custom designed apparatus which includes touchscreen mobile wireless devices such as PDAs, tablets (e.g. refers to all current and future variants, revisions and generations of the Apple IPAD, Samsung Galaxy, HP, Acer, Surface, Nook, Google Nexus, Sony, Kindle and all future tablets manufactured by these and other manufactures), related handheld devices including Apple IPOD Touch, or wearable timepieces or fob watches and other similar apparatus with WIFI and wireless capability, and remote computers and controllers with wireless connectivity that can utilize an APP (application) that can display or provides a means for recording pH and pepsin data and user events during an ambulatory study.

25. The apparatus for monitoring of pH and pepsin as recited in claim 24, wherein said catheter has separation means to restrain said self-condensing pH sensor and pepsin sensor element from directly contacting the airway mucosal membranes of a patient, said separation means located near said distal end of said catheter.

26. The apparatus for monitoring of pH and pepsin as recited in claim 25, wherein said separation means comprises a tear-drop shaped structure.

27. The apparatus for monitoring of pH and pepsin as recited in claim 24, wherein said processing receiver/data recorder is in wireless communication with said data transmitter.

28. The apparatus for monitoring of pH and pepsin as recited in claim 27, wherein said wireless communication is conducted continuously in real-time.

29. The apparatus for monitoring of pH and pepsin as recited in claim 24, wherein said processing receiver/data recorder is in continuous real-time wired communication with said data transmitter.

30. The apparatus for monitoring of pH and pepsin as recited in claim 24, further comprising a light source mounted on said catheter in close proximity to said distal end.

31. The apparatus for monitoring of pH and pepsin as recited in claim 30, wherein said light source is a light emitting diode.

32. The apparatus for monitoring of pH and pepsin as recited in claim 31, wherein said light source functions to facilitate the catheter insertion, placement and location of said pH sensor and pepsin sensor within said oropharynx area of a patient, said light source comprising a continuous or flashing light emitting diode (LED) embedded in the distal end of the catheter for providing a visual sighting means for the physician.

33. The apparatus for monitoring of pH and pepsin as recited in claim 24, further comprising a removable data storage medium, said removable data storage medium designed to communicate with said processing receiver/data recorder, said removable data storage medium further designed to store recorded pH and pepsin measurements monitored by said pH sensor and pepsin sensor over a period of time.

34. The apparatus for monitoring of pH and pepsin as recited in claim 24, further comprising a measurement system that can include a means to activate an auditory and/or visual alarm when predetermined pH values and pepsin values are detected.
